(19) United States
(12) Patent Application Publication
(10) Pub. No.: US 2009/0087769 A1
(43) Pub. Date: Apr. 2, 2009

(54) Finely divided azo dye and process for producing the same

(76) Inventors: Joachim Weber, Reinach (CH); Magali Venera, Kelkheim (DE); Karl-Heinz Schweikart, Eschborn (DE); Gerhard Wilker, Morfelden-Walldorf (DE); Frank Alfter, Bad Soden (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/919,661

(22) PCT Filed: Apr. 20, 2006
(86) PCT No.: PCT/EP2006/003625

§ 371 Date: Oct. 30, 2007

(30) Foreign Application Priority Data
May 6, 2005 (DE) 10 2005 021 159.3

(51) Int. Cl. G03G 9/06; C07C 245/06; G03G 9/08; G02B 5/23; B32B 5/16; C09D 11/00

(52) U.S. Cl. 430/108.23; 534/742; 252/586; 428/402; 106/31.65

(57) ABSTRACT

Finely divided azo dye and process for producing the same. The invention relates to a finely divided mono-azo pigment of formula (I) in the beta crystal phase (I), characterised in that at least 90 % by weight of the particles have a stoke equivalent diameter equal to or smaller than 130 nm.

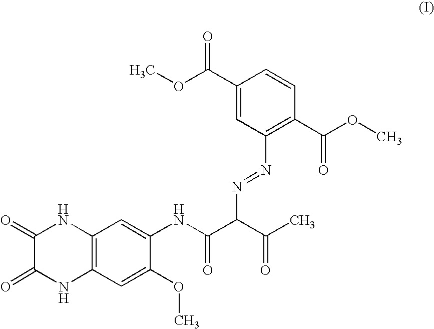

Pigments used for coloring high molecular mass organic materials are subjected to stringent requirements with regard to their performance properties, such as high color strengths, ready dispersibility, high chroma and cleanness of hue, and good light fastness and weather fastness. Universal utility for coloring various high molecular mass systems such as plastics and also for coloring aqueous and solvent-based printing inks and paints is desirable. In both paints and printing inks there is a trend toward high pigment concentrations in the grind, which is why highly pigmented paint and printing-ink concentrates or millbases with nonetheless low viscosity are called for; similarly, the viscosity of the completed paint or printing ink has to be suitable for the planned application. Printing inks are required to have a high transparency, while paint systems are desired to have impeccable overcoating fastnesses and solvent fastnesses, resistance to alkali and acid, and, in the case of metallic paint systems in particular, high transparency and brilliant hues. In the case of plastics coloration, the requirements include high bleed fastness, heat stability, and good dispersibility, as is manifested, for example, in high color strengths. Again, universal utility in various systems, such as in aqueous and solvent-based systems, is also desired.

Examples of further fields of use for pigments include electrophotographic toners and developers, liquid inks such as inkjet inks or e-inks, for example, color filters, or powder coatings, which each have their additional specific requirements.

With color filters a full-color image is produced by red, green, and blue image points using transmitted light. As well as the transmissive (or nonemissive) color filters (i.e., those using transmitted light) there are also reflective color filters, which are then able to work where appropriate with yellow, cyan, and magenta image points as well.

Among the color filters a distinction is made between AM (active matrix) and PM (passive matrix) LCD (liquid crystal display) color filters, with the TFT (thin film transistor) LCD color filters being accorded a particular significance.

Color filters can also be employed, furthermore, with MEMS (DMD) (micro-electromechanical systems, digital micro mirror devices), with e-paper, and also with further suitable display technologies.

Color filter displays find application in a very wide variety of electrooptical systems, as for example in screens of desktop monitors, in computer screens, screens of portable computers (laptops), PDAs (personal digital assistants), and also in cellphone monitors, video camera monitors, GPS (global positioning systems) monitors, and other monitors, and additionally, generally, in liquid-crystal devices and charge-coupled devices, in plasma displays or in electroluminescent and other displays. The last-mentioned displays may be, for example, active (twisted nematic) or passive (supertwisted nematic) ferroelectric displays, or light-emitting diodes, for example.

Color filters find use, moreover, in flat panel displays (flat screens), which are increasingly replacing the conventional cathode ray television screens, or which may be utilized, generally, as display panels in any desired size for fixed and moving information.

A typical LCD color filter construction may be described schematically as follows: between two glass plates there is located a thin layer with liquid crystals. Besides a number of other functional components, the upper glass plate has on its outer surface the corresponding image points, e.g., red, green, and blue (R, G, B). These image points are outlined in black for better contrast; to the outside, the R, G, B image points are protected by a suitable protective coat against environmental effects, such as scratches. The lower glass plate also contains further functional components such as, for example, ITO (indium tin oxide) and TFT (thin film transistors), which serve among other things to drive the individual image points.

If suitable light (e.g., linearly polarized light of a defined wavelength) is passed through the lower glass plate, the liquid crystal can then be driven electronically and thereby set to “light” or “dark” (or to any stage in between).

Correspondingly, the color filter image points are supplied with light and a corresponding colored image, fixed or moving, based on R, G, B, is produced to the human eye.

EP-A-0 894 831 discloses quinoxaline monoazo-acetarylide pigments, including a pigment in the formula (I)

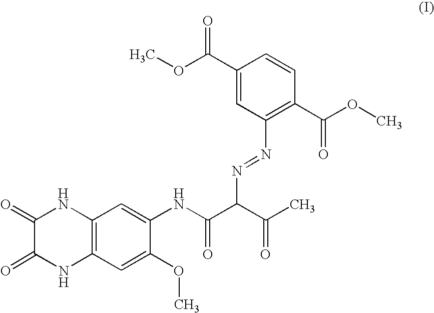

In the preparation process disclosed, in the first step, the monoazo pigment is prepared by azo coupling and is isolated in the form of a presscake. In a second step a solvent finishing operation is carried out in a dipolar-aprotic solvent. Example 1 discloses N-methylpyrrolidone as the solvent. By means of X-ray spectroscopy it is possible to show that in the course of this solvent finish there is a conversion of the original alpha crystal phase, present after azo coupling, into the beta crystal phase. The solvent finish produces a coarse monoazo pigment of the formula (I) in the beta crystal phase.

The alpha and beta crystal phases of the monoazo pigment of the formula (I) are distinguished by the following characteristic lines in the X-ray powder diagram (table 1, Cu-K-alpha radiation, twice the diffraction angle 2 theta values in degrees, with a margin of error of +/−0.2 degree, interplanar spacings d in Å, relative intensity in percent):

TABLE 1

| alpha | | | beta | | |
|---|---|---|---|---|---|
| 2 theta | d | rel. int. | 2 theta | d | rel. int. |
| 3.3 | 26.8 | 39 | 6.4 | 13.9 | 7 |
| 8.0 | 11.1 | 100 | 7.5 | 11.8 | 7 |
| 8.8 | 10.0 | 55 | 9.2 | 9.6 | 86 |
| 9.9 | 8.9 | 23 | 10.5 | 8.4 | 4 |
| 11.0 | 8.0 | 16 | 12.8 | 6.9 | 16 |
| 12.4 | 7.1 | 12 | 15.0 | 5.9 | 15 |
| 13.3 | 6.7 | 11 | 16.0 | 5.5 | 7 |
| 14.7 | 6.0 | 14 | 16.8 | 5.3 | 5 |
| 16.1 | 5.5 | 31 | 17.2 | 5.2 | 13 |
| 16.7 | 5.3 | 20 | 17.5 | 5.1 | 5 |
| 18.7 | 4.7 | 13 | 19.3 | 4.6 | 20 |
| 19.9 | 4.5 | 21 | 19.6 | 4.5 | 24 |
| 22.6 | 3.9 | 18 | 21.2 | 4.2 | 6 |
| 24.4 | 3.7 | 31 | 21.6 | 4.1 | 12 |
| 26.4 | 3.4 | 67 | 21.8 | 4.1 | 13 |
| 32.4 | 2.8 | 11 | 22.8 | 3.9 | 5 |
| 37.2 | 2.4 | 11 | 23.9 | 3.7 | 2 |
| | | | 25.3 | 3.5 | 4 |
| | | | 26.0 | 3.4 | 19 |
| | | | 26.6 | 3.4 | 100 |
| | | | 27.0 | 3.3 | 14 |
| | | | 27.8 | 3.2 | 7 |
| | | | 28.9 | 3.1 | 5 |
| | | | 30.1 | 3.0 | 4 |
| | | | 32.0 | 2.8 | 6 |
| | | | 32.3 | 2.8 | 6 |
| | | | 33.0 | 2.7 | 3 |

A coarse monoazo pigment of the formula (I) in the beta crystal phase is distinguished by high hiding power and high fluidity in paints. In various applications, however, properties are sought which cannot be achieved by a coarse pigment: for example, in an ink-jet ink, a coarse pigment leads to clogging of the nozzles and does not produce the high transparency required in ink-jet printing. Similarly, a high hiding power is prohibitive for the use of a pigment in printing inks.

There was a need for a finely divided, transparent, and readily dispersible monoazo pigment of the formula (I) in the beta crystal phase and for a process for producing it.

It has been found that the monoazo pigment of the formula (I) is obtainable, surprisingly, in its beta crystal phase and also in a finely divided, transparent, and readily dispersible form if the alpha crystal phase is subjected to salt kneading.

The invention provides a finely divided monoazo pigment of the formula (I) in the beta crystal phase, wherein at least 90% by weight of the particles have a Stokes-equivalent diameter of less than or equal to 130 nm, preferably of less than or equal to 100 nm.

The finely divided monoazo pigment of the formula (I) of the invention is notable for ready dispersibility. This is manifested when the Stokes-equivalent diameter is determined in the dispersion. The ready dispersibility and low particle diameter lead to high transparency. The transparency can be described by the contrast ratio C=Yb/Yw, with Yb being the lightness reference value via black and Yw the lightness reference value via white, and Y being one of the three tristimulus values X/Y/Z that form the basis for the various color systems. For the monoazo pigment of the formula (I) of the invention in the beta crystal phase, the values of the contrast ratio C=Yb/Nw are less than or equal to 0.22, preferably less than or equal to 0.20, more particularly less than or equal to 0.18.

The invention also provides a process for producing finely divided monoazo pigment of the formula (I) in the beta crystal phase, which comprises subjecting a monoazo pigment of the formula (I) in the alpha crystal phase to salt kneading. The alpha crystal phase of the monoazo pigment of the formula (I) can be prepared for example by azo coupling as described in EP-A-0 894 831.

Salt kneading takes place by kneading the monoazo pigment of the formula (I) in the alpha crystal phase with an organic liquid and with a crystalline salt in the form of a kneadable high-viscosity paste.

Suitable salts are salts of monovalent, divalent or trivalent metal ions, such as alkali metal ions or alkaline earth metal ions, for example, with inorganic acids, such as hydrochloric acid, sulfuric acid or phosphoric acid, or with organic acids having 1 to 6 carbon atoms, examples being formic acid and acetic acid. Preferred salts are sodium formate; sodium or calcium acetate; sodium citrate; potassium sodium tartrate; sodium, potassium, calcium, zinc or aluminum chloride; sodium or aluminum sulfate; calcium carbonate; or mixtures of these salts, and more particularly sodium chloride. The salts are generally used in a fairly large amount—for example, in at least 1 to 10 times, preferably 2 to 8 times, more particularly 3 to 6 times the amount, based on the weight of the monoazo pigment. Even larger amounts can be used, but are uneconomic.

Commercially customary salt may be coarse and may be comminuted by grinding before being used in salt kneading.

The organic liquid is employed in amounts such that the millbase forms a viscous, doughy mass. The amounts employed according to the invention are between 0.05 to 0.8 times, preferably between 0.1 to 0.4 times, in particular between 0.12 to 0.35 times, the amount, based on the weight of the monoazo pigment salt mixture. Suitable organic liquids are those in which the monoazo pigment and the salt are ideally insoluble. Examples of organic liquids of this kind are alcohols having 4 to 10 C atoms, such as butanols, such as n-butanol, isobutanol, tert-butanol, pentanols, such as n-pentanol, 2-methyl-2-butanol, hexanols, such as 2-methyl-2-pentanol, 3-methyl-3-pentanol, 2-methyl-2-hexanol, 3-ethyl-3-pentanol, octanols, such as 2,4,4-trimethyl-2-pentanol, cyclohexanol; or glycols, such as ethylene glycol, di-, tri- or tetraethylene glycol, propylene glycol, di-, tri- or tetrapropylene glycol, sorbitol or glycerol; polyglycols, such as polyethylene glycols or polypropylene glycols; ethers, such as methyl isobutyl ether, tetrahydrofuran, dimethoxyethane or dioxane; glycol ethers, such as monoalkyl ethers of ethylene glycol or propylene glycol, or diethylene glycol monoalkyl ethers, with alkyl possibly being methyl, ethyl, propyl, and butyl, examples being butyl glycols or methoxybutanol; polyethylene glycol monomethyl ethers, more particularly those having an average molar mass of 350 to 550 g/mol, and polyethylene glycol dimethyl ethers, more particularly those having an average molar mass of 250 to 500 g/mol; ketones, such as methyl isobutyl ketone, methyl ethyl ketone or cyclohexanone; aliphatic acid amides, such as formamide, dimethylformamide, N-methylacetamide or N,N-dimethylacetamide; urea derivatives, such as tetramethylurea; or cyclic carboxamides, such as N-methylpyrrolidone, valerolactam or caprolactam; esters, such as carboxylic acid C₁-C₆ glycol esters; or phthalic diesters or benzoic alkyl esters, such as benzoic acid C₁-C₄ alkyl esters or C₁-C₁₂ alkyl phthalic diesters; cyclic esters, such as caprolactone; nitriles, such as acetonitrile, aliphatic or aromatic amines, such as dimethylaniline or diethylaniline, for example; halogenated aliphatic or aromatic hydrocarbons such as carbon tetrachloride, trichloroethylene or tetrachloroethylene; or alkoxy-, nitro-, cyano- or halogen-substituted benzene, examples being anisole, nitrobenzene, dichlorobenzenes, trichlorobenzenes or benzonitrile; aromatic heterocycles, such as pyridine, morpholine, picoline or quinoline; 1,3-dimethyl-2-imidazolidinone; sulfones and sulfoxides, such as dimethyl sulfoxide and sulfolane; and also mixtures of these organic liquids. Preference is given to glycols and glycol ethers, such as ethylene glycol, diethylene glycol or butyl glycol, dimethylformamide, N-methylpyrrolidone, dimethyl sulfoxide, in particular diethylene glycol, N-methylpyrrolidone and dimethyl sulfoxide. It is possible, but generally not desired, to use a small amount of water, which ought not to be more than 25% by weight of the total liquid, including the water that can be present in the monoazo pigment employed.

For the salt kneading it is also possible to employ acids, in particular acids having a pK value of less than 4.8. Preference may be given to using phosphoric acid, carboxylic acids, such as acetic acid, dodecylbenzenesulfonic acid, and, more particularly, sulfuric acid.

The duration of kneading is guided by the target requirements and by the point in time at which the crystal phase undergoes transition, and can be from 30 minutes to 48 hours or longer; generally it is in the range from 1 to 24 hours, in particular from 2 to 8 hours.

Suitable kneaders include customary continuous and batch kneaders, these being single-arm or multiarm kneaders, preferably two-arm batch kneaders, which exert very high shearing forces on the material being kneaded. Customary blade forms are the double-trough blade (also called sigma blade or Z-blade) or the masticator blade.

The temperature during kneading should be above the melting point and below the boiling point of the organic liquid.

Kneading takes place preferably at a temperature of −20 to 150° C., in particular 50 to 120° C., with from 4 to 6 times the amount of sodium chloride salt, based on the weight of the monoazo pigment, and from 1 to 2 times the amount of diethylene glycol as organic liquid, based on the weight of the monoazo pigment.

In the course of kneading it is possible if necessary to adjust or hold constant the viscous consistency of the material being kneaded, by means of subsequent addition of organic liquid and/or salt. The consistency of the material being kneaded may alter during the kneading operation, as a result for example of evaporation of the organic liquid or as a result for example of the grinding of the monoazo pigment to smaller particle sizes.

Prior to the kneading it is possible in principle to carry out a dry preliminary grinding of monoazo pigment with salt, as is described in EP-A-1 411 091, for example.

The salt used in salt kneading and the organic liquid are preferably removed by means of an aqueous extraction carried out at acidic pH. This is generally done using acids which accelerate the dissolution of the salt employed, such as hydrochloric, sulfuric or acetic acid, for example. Typically a pH of less than 3 is set, more preferably 1 to 2, or extractive stirring is carried out in 1% to 10% strength by weight acid.

For the extraction it is also possible to add organic solvents.

Extraction can be carried out at any desired temperature, with the proviso that the medium remains liquid, and may even take place above the boiling point of the mixture, where appropriate. Since it is preferred to operate in an aqueous medium, temperatures selected are between 0 and 100° C., more particularly between 60° C. and boiling temperature.

The monoazo pigment of the formula (I) in the beta-crystal phase produced by the process of the invention can be isolated by the customary methods, such as by filtration, decanting or centrifugation, for example. Filtration is preferred. Solvents can also be removed by washing.

The monoazo pigment of the invention is employed preferably in a dried solid form, in free-flowing powder consistency, or in the form of granules, or alternatively, for example, as an aqueous presscake.

The finely divided monoazo pigment of the formula (I) of the invention may still contain traces or small amounts of the monoazo pigment of the formula (I) in the alpha crystal phase, but these are fractions of below 10% by weight, preferably below 5% by weight. Typically the crystal phase transition effected by the process of the invention is so complete that the alpha crystal phase is no longer visible in the X-ray diffraction diagram.

During salt kneading, during extraction, to the presscake, or else after the drying, it is possible to add further auxiliaries, such as, for example, surfactants, nonpigmentary and pigmentary dispersants, fillers, standardizers, resins, waxes, defoamers, antistatics, antidust agents, extenders, shading colorants, preservatives, drying retardants, rheology control additives, wetting agents, antioxidants, UV absorbers, light stabilizers, and biocides, or a combination of these.

Suitable surfactants include anionic, or anion-active, cationic, or cation-active, and nonionic or amphoteric substances, or mixtures of these agents.

By nonpigmentary dispersants are meant substances which structurally are not derived from organic pigments. They are added as dispersants either during the actual preparation of pigments, but often, also, during the incorporation of the pigments into the application media that are to be colored: for example, during the preparation of varnishes or printing inks, by dispersing the pigments into the corresponding binders.

By pigmentary dispersants are meant pigment dispersants known per se which derive from an organic pigment parent structure and are prepared by chemically modifying said parent structure, examples being saccharin-containing pigment dispersants, piperidyl-containing pigment dispersants, naphthalene- or perylene-derived pigment dispersants, pigment dispersants having functional groups which are attached to the pigment parent structure via a methylene group, pigment parent structures chemically modified with polymers, pigment dispersants containing sulfo acid, sulfonamide or sulfo acid ester groups, pigment dispersants containing ether or thioether groups, or pigment dispersants containing carboxylic acid, carboxylic ester or carboxamide groups. It is preferred to use those pigment dispersants which in structural terms derive from organic pigments with an intrinsic yellow color, as the parent structure.

In the process of the invention it is possible to use one or more pigment dispersants in a total amount of 0.1% to 25%, preferably 0.5% to 20%, more particularly 1.0% to 17.5%, by weight based on the weight of the monoazo pigment.

Anionic groups of the nonpigmentary and pigmentary dispersants, surfactants or resins used as auxiliaries may also be present in the form of salts with monovalent, divalent or trivalent ions, and in particular may be laked, using for example Ca, Mg, Ba, Sr, Mn or Al ions or using quaternary ammonium ions. By fillers and/or extenders are meant a multiplicity of substances in accordance with DIN 55943 and DIN EN 971-1, examples being the various types of talc, kaolin, mica, dolomite, lime, barium sulfate or titanium dioxide.

It was surprising and was not foreseeable that the change in crystal polymorph from alpha to beta could be brought about not only by means of the known solvent finish but also by means of a salt kneading operation. Also surprising was the fact that a new, finely divided monoazo pigment of the formula (I) in the beta crystal polymorph, with hitherto unknown properties, is formed.

The monoazo pigment of the invention is notable for outstanding coloristic and rheological properties, particularly its high flocculation stability, ready dispersibility, good rheology, high color strength, transparency, and saturation (chroma). It can be dispersed easily and up to high levels of fineness in numerous application media. Pigment dispersions of this kind exhibit outstanding rheological properties even at high levels of pigmentation of the paint or printing-ink concentrates. Other properties too, such as gloss, fastness to overcoating, solvent fastness, alkali and acid fastness, light and weather fastnesses, and high cleanness of hue, for example, are very good.

The monoazo pigment of the invention can be employed to outstanding effect in color filters. There it ensures high contrast and also satisfies the other requirements imposed in the case of color filter use, such as high temperature stability or steep and narrow absorption bands. It is also suitable, for example, for ink-jet applications, by virtue of its high color strength, and by virtue of the high storage stability at low viscosity of the ink-jet ink, which does not clog the nozzles and which exhibits high transparency.

The monoazo pigment of the invention can be employed for pigmenting high molecular mass organic materials of natural or synthetic origin, such as plastics, resins, varnishes, powder coating materials, paints, electrophotographic toners and developers, electret materials, color filters, inks, including printing inks, and seed, for example.

High molecular mass organic materials which can be pigmented with the monoazo pigment of the invention are, for example, cellulose compounds, such as, for example, cellulose ethers and cellulose esters, such as ethylcellulose, nitrocellulose, cellulose acetates or cellulose butyrates, natural binders, such as, for example, fatty acids, fatty oils, resins and their conversion products or synthetic resins, such as, for example, polycondensates, polyadducts, addition polymers and copolymers, such as, for example, amino resins, especially urea and melamine formaldehyde resins, alkyd resins, acrylic resins, phenoplasts and phenolic resins, such as novolaks or resols, urea resins, polyvinyls, such as polyvinyl alcohols, polyvinyl acetals, polyvinyl acetates or polyvinyl ethers, polycarbonates, polyolefins, such as polystyrene, polyvinyl chloride, polyethylene or polypropylene, poly(meth)acrylates and copolymers thereof, such as polyacrylic esters or polyacrylonitriles, polyamides, polyesters, polyurethanes, coumarone-indene and hydrocarbon resins, epoxy resins, unsaturated synthetic resins (polyesters, acrylates) with the different cure mechanisms, waxes, aldehyde and ketone resins, gum, rubber and its derivatives and latices, casein, silicones and silicone resins; individually or in mixtures.

It is unimportant whether the aforementioned high molecular mass organic compounds are present in the form of plastic masses or melts or in the form of spinning solutions, dispersions, varnishes, paints or printing inks. Depending on the intended use it proves advantageous to utilize the monoazo pigment of the invention in the form of a blend or in the form of prepared products or dispersions.

The present invention further provides a high molecular mass organic material comprising a coloringly effective amount of the monoazo pigment of the invention of the formula (I) in the beta-crystal phase.

Based on the high molecular mass organic material it is intended to pigment, the monoazo pigment of the invention is employed usually in an amount of 0.01% to 30% by weight, preferably 0.1% to 15% by weight.

The monoazo pigment of the invention is also suitable for use as colorants in electrophotographic toners and developers, such as, for example, one- or two-component powder toners (also called one- or two-component developers), magnetic toners, liquid toners, polymerization toners, and specialty toners.

Typical toner binders are addition-polymerization resins, polyaddition resins and polycondensation resins, such as styrene, styrene-acrylate, styrene-butadiene, acrylate, polyester, phenolic-epoxy resins, polysulfones, polyurethanes, individually or in combination, and also polyethylene and polypropylene, which may also include further ingredients, such as charge control agents, waxes or flow assistants, or may be modified subsequently with these added ingredients.

The monoazo pigment of the invention is additionally suitable for use as colorants in powders and powder coating materials, particularly in triboelectrically or electrokinetically sprayable powder coating materials which are employed to coat the surfaces of articles made, for example, from metal, wood, plastic, glass, ceramic, concrete, textile material, paper or rubber.

Moreover the monoazo pigment of the invention is suitable for use as colorants in ink-jet inks on both an aqueous and a nonaqueous basis, and also in inks which operate in accordance with the hot-melt process.

In the ink-jet inks the monoazo pigment of the invention may also be shaded with other colorants, such as organic or inorganic pigments and/or dyes, for example. In this context it is used in ink sets consisting of yellow, magenta, cyan, and black inks, comprising pigments and/or dyes as colorants. In addition it can be used in ink sets which further comprise one or more of the so-called spot colors in the colors, for example, orange, green, blue, gold, and silver.

Preference is given in this context to a set of printing inks whose black preparation preferably comprises carbon black as its colorant, more particularly a gas black or furnace black; whose cyan preparation preferably comprises a pigment from the group of the phthalocyanine, indanthrone or triarylcarbonium pigments, more particularly the Colour Index pigment Pigment Blue 15, Pigment Blue 15:1, Pigment Blue 15:2, Pigment Blue 15:3, Pigment Blue 15:4, Pigment Blue 16, Pigment Blue 56, Pigment Blue 60 or Pigment Blue 61; whose magenta preparation preferably comprises a pigment from the group of the monoazo, disazo, β-naphthol, Naphthol AS, laked azo, metal complex, benzimidazolone, anthanthrone, anthraquinone, quinacridone, dioxazine, perylene, thioindigo, triarylcarbonium or diketopyrrolopyrrole pigments, more particularly the Colour Index pigments Pigment Red 2, Pigment Red 3, Pigment Red 4, Pigment Red 5, Pigment Red 9, Pigment Red 12, Pigment Red 14, Pigment Red 38, Pigment Red 48:2, Pigment Red 48:3, Pigment Red 48:4, Pigment Red 53:1, Pigment Red 57:1, Pigment Red 112, Pigment Red 122, Pigment Red 144, Pigment Red 146, Pigment Red 147, Pigment Red 149, Pigment Red 168, Pigment Red 169, Pigment Red 170, Pigment Red 175, Pigment Red 176, Pigment Red 177, Pigment Red 179, Pigment Red 181, Pigment Red 184, Pigment Red 185, Pigment Red 187, Pigment Red 188, Pigment Red 207, Pigment Red 208, Pigment Red 209, Pigment Red 210, Pigment Red 214, Pigment Red 242, Pigment Red 247, Pigment Red 253, Pigment Red 254, Pigment Red 255, Pigment Red 256, Pigment Red 257, Pigment Red 262, Pigment Red 263, Pigment Red 264, Pigment Red 266, Pigment Red 269, Pigment Red 270, Pigment Red 272, Pigment Red 274, Pigment Violet 19, Pigment Violet 23 or Pigment Violet 32; whose yellow preparation preferably comprises a pigment from the group of the monoazo, disazo, benzimidazoline, isoindolinone, isoindoline or perinone pigments, more particularly the Colour Index pigments Pigment Yellow 1, Pigment Yellow 3, Pigment Yellow 12, Pigment Yellow 13, Pigment Yellow 14, Pigment Yellow 16, Pigment Yellow 17, Pigment Yellow 73, Pigment Yellow 74, Pigment Yellow 81, Pigment Yellow 83, Pigment Yellow 87, Pigment Yellow 97, Pigment Yellow 111, Pigment Yellow 120, Pigment Yellow 126, Pigment Yellow 127, Pigment Yellow 128, Pigment Yellow 139, Pigment Yellow 151, Pigment Yellow 154, Pigment Yellow 155, Pigment Yellow 173, Pigment Yellow 174, Pigment Yellow 175, Pigment Yellow 176, Pigment Yellow 180, Pigment Yellow 181, Pigment Yellow 191, Pigment Yellow 194, Pigment Yellow 196, Pigment Yellow 213 or Pigment Yellow 219; whose orange preparation preferably comprises a pigment from the group of the disazo, β-naphthol, Naphthol AS, benzimidazolone or perinone pigment, more particularly the Colour Index pigments Pigment Orange 5, Pigment Orange 13, Pigment Orange 34, Pigment Orange 36, Pigment Orange 38, Pigment Orange 43, Pigment Orange 62, Pigment Orange 68, Pigment Orange 70, Pigment Orange 71, Pigment Orange 72, Pigment Orange 73, Pigment Orange 74 or Pigment Orange 81; and whose green preparation preferably comprises a pigment from the group of the phthalocyanine pigments, more particularly the Colour Index pigments Pigment Green 7 or Pigment Green 36.

In addition the ink sets may further comprise shading dyes, preferably from the group of C.I. Acid Yellow 17 and C.I. Acid Yellow 23; C.I. Direct Yellow 86, C.I. Direct Yellow 98 and C.I. Direct Yellow 132; C.I. Reactive Yellow 37; C.I. Pigment Yellow 17, C.I. Pigment Yellow 74, C.I. Pigment Yellow 83, C.I. Pigment Yellow 97, C.I. Pigment Yellow 120, C.I. Pigment Yellow 139, C.I. Pigment Yellow 151, C.I. Pigment Yellow 155 and C.I. Pigment Yellow 180; C.I. Direct Red 1, C.I. Direct Red 11, C.I. Direct Red 37, C.I. Direct Red 62, C.I. Direct Red 75, C.I. Direct Red 81, C.I. Direct Red 87, C.I. Direct Red 89, C.I. Direct Red 95 and C.I. Direct Red 227; C.I. Acid Red 1, C.I. Acid Red 8, C.I. Acid Red 80, C.I. Acid Red 81, C.I. Acid Red 82, C.I. Acid Red 87, C.I. Acid Red 94, C.I. Acid Red 115, C.I. Acid Red 131, C.I. Acid Red 144, C.I. Acid Red 152, C.I. Acid Red 154, C.I. Acid Red 186, C.I. Acid Red 245, C.I. Acid Red 249 and C.I. Acid Red 289; C.I. Reactive Red 21, C.I. Reactive Red 22, C.I. Reactive Red 23, C.I. Reactive Red 35, C.I. Reactive Red 63, C.I. Reactive Red 106, C.I. Reactive Red 107, C.I. Reactive Red 112, C.I. Reactive Red 113, C.I. Reactive Red 114, C.I. Reactive Red 126, C.I. Reactive Red 127, C.I. Reactive Red 128, C.I. Reactive Red 129, C.I. Reactive Red 130, C.I. Reactive Red 131, C.I. Reactive Red 137, C.I. Reactive Red 160, C.I. Reactive Red 161, C.I. Reactive Red 174 and C.I. Reactive Red 180. Ink-jet inks generally contain a total of 0.5% to 15% by weight, preferably 1.5% to 8% by weight (reckoned on a dry basis), of the monoazo pigment of the invention. Microemulsion inks are based on organic solvents, water, and, where appropriate, an additional hydrotropic substance (interface mediator). Microemulsion inks contain generally 0.5% to 15% by weight, preferably 1.5% to 8% by weight, of the monoazo pigment of the invention, 5% to 99% by weight of water, and 0.5% to 94.5% by weight of organic solvent and/or hydrotropic compound.

“Solvent based” ink-jet inks contain preferably 0.5% to 15% by weight of the monoazo pigment of the invention, 85% to 99.5% by weight of at least one organic solvent and/or hydrotropic compounds.

Hot-melt inks are based usually on waxes, fatty acids, fatty alcohols or sulfonamides which are solid at room temperature and liquefy on heating, the preferred melting range being between about 60° C. and about 140° C. Hot-melt ink-jet inks are composed, for example, essentially of 20% to 90% by weight of wax and 1% to 10% by weight of the monoazo pigment of the invention. They may further include 0 to 20% by weight of an additional polymer (as “dye dissolver”), 0 to 5% by weight of dispersing assistant, 0 to 20% by weight of viscosity modifier, 0 to 20% by weight of plasticizer, 0 to 10% by weight of tack additive, 0 to 10% by weight of transparency stabilizer (which prevents, for example, crystallization of the waxes), and 0 to 2% by weight of antioxidant.

Additionally the monoazo pigment of the invention is also suitable for use as colorants for color filters, both for additive and for subtractive color generation, such as, for example, in electrooptical systems such as television screens, LCDs (liquid crystal displays), charge-coupled devices, plasma displays or electroluminescent displays, which may in turn be active (twisted nematic) or passive (supertwisted nematic) ferroelectric displays or light-emitting diodes, and also as colorants for electronic inks (or e-inks) or electronic paper (e-paper).

In the production of color filters, both reflective and transparent color filters, pigments are applied in the form of a paste or as pigmented photoresists in suitable binders (acrylates, acrylic esters, polyimides, polyvinyl alcohols, epoxides, polyesters, melamines, gelatins, caseins) to the respective LCD components (e.g., TFT-LCD—Thin Film Transistor Liquid Crystal Displays or, e.g., ((S) TN-LCD—(Super) Twisted Nematic-LCD). Besides high thermal stability, high pigment purity is a prerequisite for a stable paste and/or a pigmented photoresist. Furthermore, the pigmented color filters can also be applied by ink-jet printing processes or other suitable printing processes.

With regard to the color filter materials there are very particular requirements imposed on the colorants employed.

The principal technical parameters which must be met are as follows:
high thermal stability: during the manufacturing operation of a color filter, the individual applied layers are heated, so that the monoazo pigment of the formula (I) in the beta-crystal phase must withstand temperatures up to 300° C. for up to 1 hour;
ready dispersibility in color filter systems;
steep and narrow absorption bands of each applied color filter layer;
high contrast;
high and stable viscosity in the color filter medium: too high a viscosity prevents the liquid being distributed uniformly on the glass substrate and detracts, as a result, from the quality of the image;
ecotoxicological benignancy in processing;
nonflocculating behavior;
a very smooth (not rough) surface of the applied (pigmented) color filters;
acid resistance (for etching processes, for example);
solvent fastness.

The invention further provides a color filter comprising the monoazo pigment of the invention in a coloringly effective amount.

The yellow hue of the monoazo pigment of the invention is highly suitable for the shading, particularly of the red and green hues, of the color filter color set red-green-blue (R, G, B). These three colors are present as separate color points alongside one another, and when backlit produce a full-color image.

Typical colorants for the red color point are pyrrolopyrrole, quinacridone and azo pigments, such as P.R. 254, P.R. 209, P.R. 175 and P.O. 38, for example, individually or mixed.

For the green color point, phthalocyanine colorants are typically employed, such as P.G. 36 and P.G. 7, for example.

For the blue color point, use is made typically of unhalogenated phthalocyanine colorants or phthalocyanine colorants with only low levels of halogenation, such as P.B. 15:6, for example.

As and when required, the respective color points may also be admixed with further colors for the purpose of shading.

In order to assess the properties of the pigments in the paint sector, in water-free, solvent-based varnish systems, a selection was made, from among the multiplicity of known varnishes, of an alkyd-melamine resin varnish based on a medium-oil alkyd resin and on a butanol-etherified melamine resin (AM).

The coloristic properties were determined in accordance with DIN 55986. The viscosity was determined following dilution of the millbase to the final pigment concentration, using the Rossman viscospatula type 301 from Erichsen.

In the examples which follow, parts and percentages are by weight unless indicated otherwise.

### EXAMPLE 1

187.5 g of monoazo pigment of the formula (I) in the alpha polymorph, prepared in accordance with example 1 of EP-A-0 894 831, the as-synthesized presscake being washed salt-free and dried, without the treatment in N-methylpyrrolidone, are kneaded in a kneader with Sigma blades at 85° C. with 1125 g of micronized sodium chloride and 330 ml of diethylene glycol for 8 hours. The kneaded material is stirred in 8 liters of 5% strength by weight sulfuric acid at 40° C. for 2 hours, the suspension is filtered with suction, the presscake is washed salt-free with water and dried at 80° C., and the granules are pulverized. This gives 180 g of monoazo pigment of the formula (I) in the beta polymorph. In the X-ray powder diagram the alpha polymorph is no longer detectable. 90% by weight of the particles have a Stokes-equivalent diameter of less than or equal to 93 nm.

### COMPARATIVE EXAMPLE 1

If the monoazo pigment of the formula (I) in the beta crystal phase is prepared according to example 1 of EP-A-0 894 831, the result is a coarse monoazo pigment; 90% by weight of the particles have a Stokes-equivalent diameter of less than or equal to 347 nm.

### EXAMPLE 2

15 g of monoazo pigment of the formula (I) in the alpha polymorph, prepared in accordance with example 1 of EP-A-0 894 831, the as-synthesized presscake being washed salt-free and dried, without the treatment in N-methylpyrrolidone, are kneaded in a kneader with Sigma blades at 25° C. with 90 g of micronized sodium chloride and 35 ml of dimethyl sulfoxide for 2 hours.

The kneaded material is stirred in 8 liters of 5% strength by weight sulfuric acid at 40° C. for 2 hours, the suspension is filtered with suction, the presscake is washed salt-free with water and dried at 80° C., and the granules are pulverized. This gives 9 g of monoazo pigment of the formula (I) in the beta polymorph. In the X-ray powder diagram the alpha polymorph is no longer detectable. 90% by weight of the particles have a Stokes-equivalent diameter of less than or equal to 127 nm.

Description of the method of determining the Stokes-equivalent particle size diameter:

The Stokes-equivalent particle size diameter is determined using the color filter pastes described below. The diluted color filter pastes are subjected to measurement by customary methods for determining particle size distribution by photosedimentometry (cf. Herbst and Hunger, Industrial Organic Pigments, VCH 2004, pp. 31-33 and 37-41, and S. T. Fitzpatrick, Polymer News, 1999, vol. 24. No. 2. pp. 42-50) in the DC24000 disk centrifuge from CPS Instruments, Inc., Stuart, Fla. 34997, USA. Dilution and measurement take place with and in an organic medium adapted to the color filter pastes. The DC24000 is operated with a rotary speed of 20 000 min⁻¹. When this speed is reached, the spin fluid is introduced into the disk. A density gradient is generated in the spin fluid by addition of 10-25% (v/v) of bis(3,5,5-trimethylhexyl) phthalate in “CB”. “CB” is a mixture of cyclohexanone and n-butyl acetate (2:3 by weight). 0.1 ml of the color filter pastes diluted 1:100 with “CB” and sonicated for 2 minutes in an ultrasound bath are applied to 13.8 ml of spin fluid. The parameter measured is the absorbance of blue light (wavelength 470 nm) deriving from the scattering and absorption of the particles which cross the beam of light close to the outer disk edge. The particle size distribution by volume fractions is calculated by the CPS instrument software, with the aid of the Mie theory. For this purpose the complex refractive index of the pigment is required. This index was determined by ellipsometry on pressed tablets of the pigment powder. The pigment particle density, likewise required, was determined using a helium gas pycnometer (AccuPyc 1330 from Micromeritics). The centrifuge is calibrated with a particle size standard immediately prior to each measurement of a sample. The standard used was monocrystalline diamond powder (average particle size 0.52 μm, very narrow particle size distribution).

### APPLICATION EXAMPLE 1

In the AM system, the monoazo pigment produced according to example 1 gives strongly colored, transparent, greenish yellow coatings. The viscosity of the coating material is low; the gloss is high. In comparison to a pigment produced in accordance with EP-A-0 894 831, example 1, the color strength is substantially higher and the masstone is substantially more transparent.

### APPLICATION EXAMPLE 2

Test method for color filters

Production of a test color filter:

First of all a color filter paste is prepared, which is composed of pigment composition, binder, solvent, and dispersing assistant in accordance with the following formula:

| | |
|---|---|
| 77% by weight | 1-methoxy-2-propyl acetate |
| 10% by weight | styrene-acrylic polymer |
| 10% by weight | pigment; and |
| 3% by weight | dispersing assistant. |

The above mixture is dispersed in a paint shaker for 2 hours with zirconium beads (Ø 0.5-0.7 mm). The dispersion is subsequently filtered. The resulting color filter paste is applied by a spin coater to a glass substrate in order to produce a color filter film. The transparency, coloristic values, heat stability, and contrast are determined on this color filter film.

The transmittance of the coated glass substrate is determined spectrophotometrically in the application range of 400-700 nm. The coloristic values are described using the CIE color triangle (xyY values): x here describes the blue-red axis, y the blue-green axis, and Y the brightness.

The viscosity is determined on the above-described color filter paste using a rotational viscometer at a temperature of 23° C.±0.5° C. and at a shear rate of 60 s⁻¹.

The heat stability is described by the delta E value; the delta E value is determined in accordance with DIN 6174; it describes the total color distance and can be calculated from the x, y, Y values. Following measurement of the transmittance, the coated glass substrate is heated at 80° C. for 10 minutes. Thereafter the transmittance is measured and the delta E is calculated. The coated glass substrate is then heated at 250° C. for 1 h, and again a delta E value is determined. Moreover, using the color filter paste, a masstone drawdown and, after dilution with a white paste, a white reduction drawdown are prepared by knife coating, and their coloristic properties are assessed. Testing for color filters with the pigment produced according to example 1:

A color filter paste is produced. The viscosity of the color filter paste is as follows: η=7 mPa.s.

Then 3 ml of the color filter paste are pipetted and applied to a glass substrate by means of a spin coater at a rotary speed of 2500 rpm for 20 s. The coloristic properties of the color filter film were then determined spectrophotometrically.

Coloristic values:

| x | y | Y |
|---|---|---|
| 0.389 | 0.448 | 83.56 |

Transmittance values:

| Wavelength | 400 nm | 410 nm | 420 nm | 430 nm | 440 nm | 450 nm |
|---|---|---|---|---|---|---|
| Transmittance (%) | 2.5 | 3.6 | 5.7 | 9.2 | 14.4 | 22.0 |
| Wavelength | 460 nm | 470 nm | 480 nm | 490 nm | 500 nm | |
| Transmittance (%) | 32.7 | 43.4 | 56.5 | 68.3 | 77.2 | |
| Wavelength | 510 nm | 520 nm | 530 nm | 540 nm | 550 nm | 560 nm |
| Transmittance (%) | 82.9 | 86.2 | 88.1 | 89.2 | 89.7 | 89.7 |
| Wavelength | 570 nm | 580 nm | 590 nm | 600 nm | 610 nm | |
| Transmittance (%) | 89.5 | 89.2 | 89 | 88.8 | 88.8 | |
| Wavelength | 620 nm | 630 nm | 640 nm | 650 nm | 660 nm | 670 nm |
| Transmittance (%) | 88.9 | 89 | 89.4 | 89.8 | 90.3 | 90.7 |
| Wavelength | 680 nm | 690 nm | 700 nm | | | |
| Transmittance (%) | 91.0 | 91.2 | 91.3 | | | |

The heat stability is good.

The drawdowns exhibit high transparency and color strength and a clean hue.

### APPLICATION EXAMPLE 3

Production of colorant formulations for ink-jet printing:

The pigment is pasted up together with the dispersants outlined below, the organic solvent, and the other additives, in deionized water, and then the paste is homogenized and pre-dispersed using a dissolver. Subsequent fine dispersion takes place using a bead mill, with grinding, accompanied by cooling, taking place until the desired particle size distribution of the pigment particles was accomplished. Thereafter the dispersion is adjusted with deionized water to the desired final pigment concentration.

The colorant formulation described in the example below was produced by the process described above, using the following constituents in the stated amounts such that 100 parts of the colorant formulation are formed, parts being by weight.

Colorant formulation 1 for ink-jet:

| | |
|---|---|
| 20 parts | pigment prepared according to example 1 |
| 2.5 parts | acrylate resin, Na salt (dispersant) |
| 1.2 parts | polyethylene glycol alkyl ether, Na salt (dispersant) |
| 7.5 parts | propylene glycol |
| 0.2 part | preservative |
| remainder | water |

Testing of the printing properties of the colorant preparation:

To assess the printing properties, a test ink was prepared from the above colorant formulation 1, and its printability was investigated using a thermal ink-jet printer.

The test ink was prepared by first finely filtering the colorant formulation 1 through a 1 μm filter to remove grinding media attritus and any coarse fractions. Thereafter the filtered colorant formulation was diluted with water and admixed with further low molecular mass alcohols and polyols, the pigment content being adjusted to 5% by weight relative to the ink (100% by weight).

An HP 960C (Hewlett Packard) printer was used to print test images on commercially customary standard papers (copying papers) and specialty papers (premium quality) from Hewlett Packard. Assessment in terms of the quality and grade of the printed image was made by means of visual inspection.

The test ink prepared from colorant formulation 1 showed very good printed characteristics. A particular outcome was the high reliability of the test ink in the course of printing (very good start-of-print behavior, no nozzle clogging) and a very uniform printed image of excellent quality on the various papers used.

1. Finely divided monoazo pigment particles of the formula (I) in the beta crystal phase, 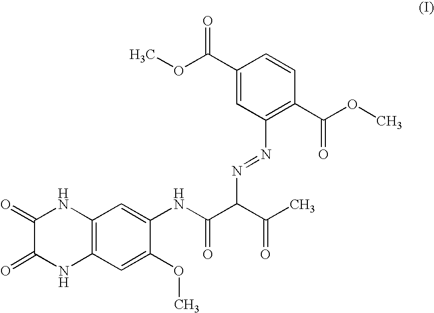
wherein at least 90% by weight of the particles have a Stokes-equivalent diameter of less than or equal to 130 nm.

2. The monoazo pigment particles as claimed in claim 1, wherein at least 90% by weight of the particles have a Stokes-equivalent diameter of less than or equal to 100 nm.

3. The monoazo pigment particles as claimed in claim 1, wherein the contrast ratio C=Yb/Yw is less than or equal to 0.22.

4. The monoazo pigment particles as claimed in claim 1, wherein the contrast ratio C=Yb/Yw is less than or equal to 0.20.

5. A process for producing monoazo pigment particles as claimed in claim 1, comprising the step of subjecting a monoazo pigment particles of the formula (I) in the alpha crystal phase to salt kneading.

6. The process as claimed in claim 5, wherein the monoazo pigment particles of the formula (I) in the alpha crystal phase are kneaded with an organic liquid and with a crystalline salt in the form of a kneadable paste of high viscosity.

7. The process as claimed in claim 6, wherein the organic liquid is selected from the group consisting of glycol, glycol ether, dimethylformamide, N-methylpyrrolidone and dimethyl sulfoxide.

8. The process as claimed in claim 6, wherein the organic liquid is diethylene glycol.

9. A high molecular mass organic material of natural or synthetic origin pigmented with monoazo pigment particles according to claim 1, wherein the high molecular mass organic material of natural or synthetic origin is selected from the group consisting of electrophotographic toners, electrophotographic developers, electret materials, color filters, inks, printing inks, and seed.

10. A high molecular mass organic material comprising a coloringly effective amount of the monoazo pigment particles as claimed in claim 1.

11. A color filter comprising a coloringly effective amount of the monoazo pigment particles as claimed in claim 1.
